(19) United States
(12) Patent Application Publication
(10) Pub. No.: US 2020/0029937 A1
(43) Pub. Date: Jan. 30, 2020

(54) ULTRASOUND DIAGNOSIS APPARATUS AND IMAGE PROCESSING METHOD

(71) Applicant: CANON MEDICAL SYSTEMS CORPORATION, Otawara-shi (JP)

(72) Inventors: Ryota OSUMI, Nasushiobara (JP); Muneki KATAGUCHI, Nasushiobara (JP); Tomohisa IMAMURA, Kawasaki (JP)

(73) Assignee: CANON MEDICAL SYSTEMS CORPORATION, Otawara-shi (JP)

(21) Appl. No.: 16/506,727

(22) Filed: Jul. 9, 2019

(30) Foreign Application Priority Data
Jul. 26, 2018 (JP) 2018-140470

(51) Int. Cl. A61B 8/08; A61B 8/00; A61B 8/06

(52) U.S. Cl. CPC A61B 8/5207; A61B 8/463; A61B 8/4444; A61B 8/488; A61B 8/06

(57) ABSTRACT

An ultrasound diagnosis apparatus according to an embodiment includes processing circuitry. The processing circuitry is configured to generate an ultrasound image on the basis of a result of an ultrasound scan performed on a region including a part of a subject. The processing circuitry is configured to obtain a schematic image schematically indicating the part of the subject. The processing circuitry is configured to cause a display to display the schematic image and either the ultrasound image or an image based on the ultrasound image, in such a manner that the orientation of the subject included in either the ultrasound image or the image based on the ultrasound image and the orientation of the subject indicated in the schematic image are close to each other, on the basis of an analysis result from an analysis performed on either the ultrasound image or the image based on the ultrasound image.

### CROSS-REFERENCE TO RELATED APPLICATIONS

This application is based upon and claims the benefit of priority from Japanese Patent Application No. 2018-140470, filed on Jul. 26, 2018; the entire contents of which are incorporated herein by reference.

### FIELD

Embodiments described herein relate generally to an ultrasound diagnosis apparatus and an image processing method.

### BACKGROUND

Ultrasound images can be used for checking growth of fetuses. For example, by using an ultrasound image, an ultrasound diagnosis apparatus is capable of measuring parameters such as the biparietal diameter (BPD), the head circumference (HC), the abdominal circumference (AC), the femur length (FL), the humerus length (HL), and the like of a fetus. By using these parameters, the ultrasound diagnosis apparatus is capable of calculating an estimated fetal weight (EFW).

In relation to this, for the purpose of guiding operations performed by an operator who performs the measuring process, the ultrasound diagnosis apparatus may cause a display to display an ultrasound image rendering a region including a part of the fetus and a schematic image schematically indicating the part of the fetus, so as to be kept in correspondence with each other.

### BRIEF DESCRIPTION OF THE DRAWINGS

FIG. 1 is a block diagram illustrating an exemplary configuration of an ultrasound diagnosis apparatus according to a first embodiment;

FIG. 2 is a flowchart illustrating a procedure in a process performed by the ultrasound diagnosis apparatus according to the first embodiment;

FIG. 3 is a drawing for explaining examples of processes performed by an image obtaining function of the ultrasound diagnosis apparatus according to the first embodiment;

FIG. 4 is another drawing for explaining the examples of the processes performed by the image obtaining function of the ultrasound diagnosis apparatus according to the first embodiment;

FIG. 5 is yet another drawing for explaining the examples of the processes performed by the image obtaining function of the ultrasound diagnosis apparatus according to the first embodiment;

FIG. 6 is a drawing for explaining an example of a process performed by a schematic image obtaining function of the ultrasound diagnosis apparatus according to the first embodiment;

FIG. 7 is a drawing for explaining examples of processes performed by an analyzing function of the ultrasound diagnosis apparatus according to the first embodiment;

FIG. 6 is another drawing for explaining the examples of the processes performed by the analyzing function of the ultrasound diagnosis apparatus according to the first embodiment;

FIG. 9 is yet another drawing for explaining the examples of the processes performed by the analyzing function of the ultrasound diagnosis apparatus according to the first embodiment;

FIG. 10 is yet another drawing for explaining the examples of the processes performed by the analyzing function of the ultrasound diagnosis apparatus according to the first embodiment;

FIG. 11 is a drawing for explaining examples of processes performed by a display controlling function of the ultrasound diagnosis apparatus according to the first embodiment;

FIG. 12 is another drawing for explaining the examples of the processes performed by the display controlling function of the ultrasound diagnosis apparatus according to the first embodiment;

FIG. 13 is yet another drawing for explaining the examples of the processes performed by the display controlling function of the ultrasound diagnosis apparatus according to the first embodiment;

FIG. 14 is yet another drawing for explaining the examples of the processes performed by the display controlling function of the ultrasound diagnosis apparatus according to the first embodiment;

FIG. 15 is a flowchart illustrating a procedure in a parameter measuring process performed by the ultrasound diagnosis apparatus according to the first embodiment; and

FIG. 16 is a drawing for explaining an example of a process performed by an estimating function of the ultrasound diagnosis apparatus according to the first embodiment.

### DETAILED DESCRIPTION

An ultrasound diagnosis apparatus according to an embodiment includes processing circuitry. The processing circuitry is configured to generate an ultrasound image on the basis of a result of an ultrasound scan performed on a region including a part of a subject. The processing circuitry is configured to obtain a schematic image schematically indicating the part of the subject. The processing circuitry is configured to cause a display to display the schematic image and either the ultrasound image or an image based on the ultrasound image, in such a manner that the orientation of the subject included in either the ultrasound image or the image based on the ultrasound image and the orientation of the subject indicated in the schematic image are close to each other, on the basis of an analysis result from an analysis performed on either the ultrasound image or the image based on the ultrasound image.

Exemplary embodiments of an ultrasound diagnosis apparatus and an image processing method will be explained below, with reference to the accompanying drawings. Possible embodiments are not limited to the embodiments described below. Further, the explanation of each of the embodiments is, in principle, similarly applicable to any other embodiment.

### First Embodiment

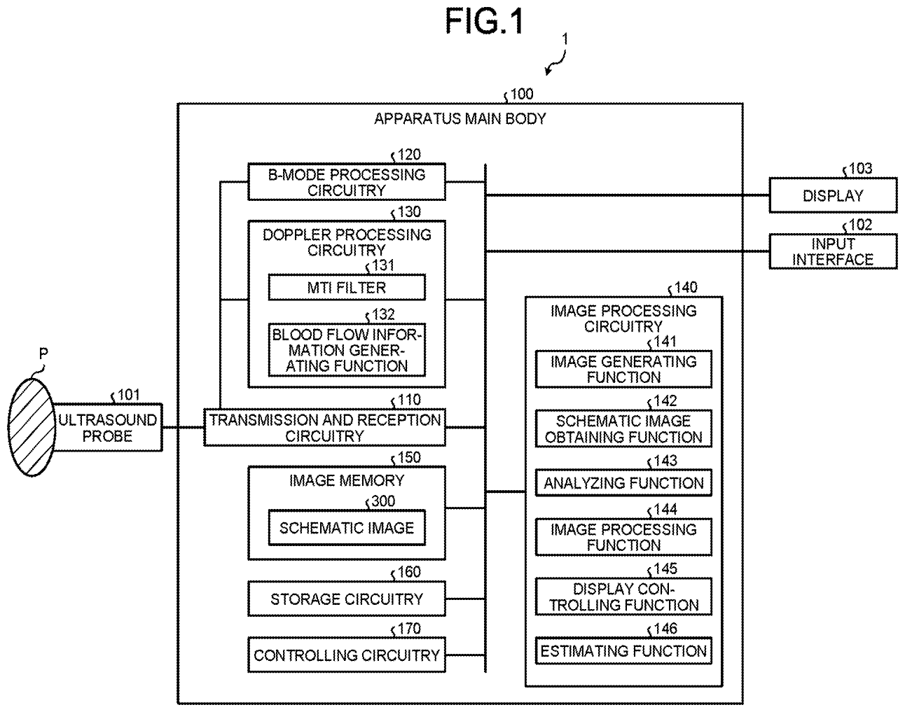

FIG. 1 is a block diagram illustrating an exemplary configuration of an ultrasound diagnosis apparatus 1 according to the first embodiment. As illustrated in FIG. 1, the ultrasound diagnosis apparatus 1 according to the first embodiment includes an apparatus main body 100, an ultrasound probe 101, an input interface 102, and a display 103. The ultrasound probe 101, the input interface 102, and the display 103 are connected to the apparatus main body 100.

The ultrasound probe 101 is configured to perform an ultrasound wave transmission/reception process (an ultrasound scan). For example, the ultrasound probe 101 is brought into contact with the body surface of a subject (hereinafter “patient”) P (the abdomen of a pregnant woman) and is configured to perform the ultrasound wave transmission/reception process on a region including at least part of a fetus in the uterus of the pregnant woman. The ultrasound probe 101 includes a plurality of piezoelectric transducer elements. Each of the plurality of piezoelectric transducer elements is a piezoelectric element having a piezoelectric effect for converting an electric signal (pulse voltage) and mechanical vibration (vibration from sound) to and from each other and is configured to generate an ultrasound wave on the basis of a drive signal (an electric signal) supplied thereto from the apparatus main body 100. The generated ultrasound waves are reflected on a plane of unmatched acoustic impedance in the body of the patient P and are received by the plurality of piezoelectric transducer elements as reflected-wave signals (electrical signals) including a component scattered by a scattering member in a tissue, and the like. The ultrasound probe 101 is configured to forward the reflected-wave signals received by the plurality of piezoelectric transducer elements to the apparatus main body 100.

In the present embodiment, as the ultrasound probe 101, an ultrasound probe in any form may be used, such as a one-dimensional (1D) array probe including the plurality of piezoelectric transducer elements arranged one-dimensionally in a predetermined direction, a two-dimensional (2D) array probe in which the plurality of piezoelectric transducer elements are two-dimensionally arranged in a matrix formation, or a mechanical four-dimensional (4D) probe configured to scan a three-dimensional region by mechanically swinging the plurality of piezoelectric transducer elements arranged one-dimensionally.

The input interface 102 includes a mouse, a keyboard, a button, a panel switch, a touch command screen, a foot switch, a wheel, a trackball, a joystick, and/or the like and is configured to receive various types of setting requests from an operator of the ultrasound diagnosis apparatus 1 and to transfer the received various types of setting requests to the apparatus main body 100.

The display 103 is configured to display a Graphical User Interface (GUI) used by the operator of the ultrasound diagnosis apparatus 1 for inputting the various types of setting requests through the input interface 102 and to display ultrasound image data generated by the apparatus main body 100 and the like.

The apparatus main body 100 is an apparatus configured to generate the ultrasound image data on the basis of the reflected-wave signals received by the ultrasound probe 101. The ultrasound image data generated by the apparatus main body 100 may be two-dimensional ultrasound image data generated on the basis of two-dimensional reflected-wave signals or may be three-dimensional ultrasound image data generated on the basis of three-dimensional reflected-wave signals.

As illustrated in FIG. 1, the apparatus main body 100 includes, for example, a transmission and reception circuitry 110, a B-mode processing circuitry 120, a Doppler processing circuitry 130, an image processing circuitry 140, an image memory 150, a storage circuitry 160, and a controlling circuitry 170. The transmission and reception circuitry 110, the B-mode processing circuitry 120, the Doppler processing circuitry 130, the image processing circuitry 140, the image memory 150, the storage circuitry 160, and the controlling circuitry 170 are communicably connected to one another.

The transmission and reception circuitry 110 is configured to control the transmission of the ultrasound waves by the ultrasound probe 101. For example, on the basis of an instruction from the controlling circuitry 170, the transmission and reception circuitry 110 is configured to apply the abovementioned drive signal (a drive pulse) to the ultrasound probe 101 with timing to which a predetermined transmission delay period is applied for each of the transducer elements. With this arrangement, the transmission and reception circuitry 110 causes the ultrasound probe 101 to transmit an ultrasound beam obtained by converging the ultrasound waves in the form of a beam.

Further, the transmission and reception circuitry 110 is configured to control the reception of the reflected-wave signals by the ultrasound probe 101. As explained above, the reflected-wave signals are signals obtained as a result of the ultrasound waves transmitted from the ultrasound probe 101 being reflected in the tissue in the body of the patient P. For example, on the basis of an instruction from the controlling circuitry 170, the transmission and reception circuitry 110 performs an adding process by applying predetermined delay periods to the reflected-wave signals received by the ultrasound probe 101. As a result, reflected components from a direction corresponding to reception directionality of the reflected-wave signals are emphasized. Further, the transmission and reception circuitry 110 converts the reflected-wave signals resulting from the adding process into an In-phase signal (an I signal) and a Quadrature-phase signal (a Q signal) that are in a baseband. Further, the transmission and reception circuitry 110 sends the I signal and the Q signal (hereinafter, “IQ signals”) as reflected-wave data, to the B-mode processing circuitry 120 and to the Doppler processing circuitry 130. In this situation, the transmission and reception circuitry 110 may send the reflected-wave signals resulting from the adding process to the B-mode processing circuitry 120 and to the Doppler processing circuitry 130, after converting the reflected-wave signals into Radio Frequency (RF) signals. The IQ signals and the RB signals are signals (the reflected-wave data) including phase information.

The B-mode processing circuitry 120 is configured to perform various types of signal processing processes on the reflected-wave data generated by the transmission and reception circuitry 110 from the reflected-wave signals. The B-mode processing circuitry 120 is configured to generate data (B-mode data) in which the signal intensity corresponding to each sampling point (measuring points) is expressed by a degree of brightness, by performing a logarithmic amplification, an envelope detecting process, or the like on the reflected-wave data received from the transmission and reception circuitry 110. The B-mode processing circuitry 120 is configured to send the generated B-mode data to the image processing circuitry 140.

Further, the B-mode processing circuitry 120 is configured to perform a signal processing process to implement a harmonic imaging process by which a harmonic component is rendered in a picture. Known examples of the harmonic imaging process include Contrast Harmonic Imaging (CHI) and Tissue Harmonic Imaging (THI) processes. Further, known examples of scanning methods used for the contrast harmonic imaging and tissue harmonic imaging processes include an Amplitude Modulation (AM) method, a Phase Modulation (PM) method called “a pulse subtraction method” or “a pulse inversion method”, and an AMPM method with which it is possible to achieve both advantageous effects of the AM method and advantageous effects of the PM method, by combining together the method and the PM method.

From the reflect-wave data generated from the reflected-wave signals by the transmission and reception circuitry 110, the Doppler processing circuitry 130 is configured to generate, as Doppler data, data obtained by extracting motion information of moving members based on the Doppler effect at sampling points within a scanned region. In this situation, the motion information of the moving members may be average velocity values, dispersion values, power values, and the like of the moving members. Examples of the moving member include, for instance, blood flows, a tissue such as the cardiac wall, and a contrast agent. The Doppler processing circuitry 130 is configured to send the generated Doppler data to the image processing circuitry 140.

For example, when the moving member is a blood flow, the motion information of the blood flow is information (blood flow information) such as an average velocity value, a dispersion value, a power value, and the like of the blood flow. It is possible to obtain the blood flow information by implementing a color Doppler method, for example.

According to the color Doppler method, at first, the ultrasound wave transmission/reception process is performed multiple times on mutually the same scanning line. Subsequently, by using a Moving Target Indicator (MTI) filter, from among signals expressing a data sequence of pieces of reflected-wave data in mutually the same position (mutually the same sampling point), signals in a specific frequency band are passed, while signals in other frequency bands are attenuated. In other words, signals (a clutter component) derived from stationary or slow-moving tissues are suppressed. With this arrangement, from among the signals expressing the data sequence of the pieces of reflected-wave data, the blood flow signal related to the blood flow is extracted. Further, according to the color Doppler method, from the extracted blood flow signal, the blood flow information such as the average velocity value, the dispersion value, the power value, and the like of the blood flow is estimated, so as to generate the estimated blood flow information as the Doppler data.

When using the abovementioned color Doppler method, the Doppler processing circuitry 130 includes, as illustrated in FIG. 1, an MTI filter 131 and a blood flow information generating function 132.

By using a filter matrix, the MTI filter 131 is configured to output a data sequence obtained by extracting the signal (the blood flow signal) in which the clutter component is suppressed, from the data sequence of the pieces of reflected-wave data in mutually the same position (the same sampling point). As the MTI filter 131, it is possible to use, for example, a filter having a fixed coefficient such as a Butterworth Infinite Impulse Response (IIR) filter, a polynomial regression filter, or the like or a filter (an adaptive filter) that varies a coefficient thereof in accordance with an input signal, by using an eigenvector or the like.

The blood flow information generating unit 132 is configured to estimate the blood flow information such as the average velocity value, the dispersion value, the power value, and the like of the blood flow on the basis of the blood flow signal, by performing a calculation such as an autocorrelation calculation on the data sequence (the blood flow signal) output by the MTI filter 131, and to generate the estimated blood flow information as Doppler data. The blood flow information generating function 132 is configured to send the generated Doppler data to the image processing circuitry 140.

The image processing circuitry 140 is configured to perform image data (ultrasound image data) generating processes and various types of image processing process on image data. For example, from two-dimensional B-mode data generated by the B-mode processing circuitry 120, the image processing circuitry 140 generates two-dimensional B-mode image data in which intensities of the reflected waves are expressed with brightness levels. Further, from two-dimensional Doppler data generated by the Doppler processing circuitry 130, the image processing circuitry 140 generates two-dimensional Doppler image data in which the blood flow information is rendered as a picture. The two-dimensional Doppler image data may be velocity image data expressing the average velocity of the blood flow, dispersion image data expressing the dispersion value of the blood flow, power image data expressing the power of the blood flow, or image data combining any of these types of image data together. As the Doppler image data, the image processing circuitry 140 is configured to generate color Doppler image data in which the blood flow information such as the average velocity, the dispersion value, the power, and/or the like of the blood flow are displayed in color and to generate Doppler image data in which a piece of blood flow information is displayed by using a gray scale.

In this situation, generally speaking, the image processing circuitry 140 converts (by performing a scan convert process) a scanning line signal sequence from an ultrasound scan into a scanning line signal sequence in a video format used by, for example, television and generates display-purpose ultrasound image data. More specifically, the image processing circuitry 140 generates the display-purpose ultrasound image data by performing a coordinate transformation process compliant with the ultrasound scanning mode used by the ultrasound probe 101. Further, as various types of image processing processes besides the scan convert process, the image processing circuitry 140 performs, for example, an image processing process (a smoothing process) to re-generate an average brightness value image, an image processing process (an edge enhancement process) that uses a differential filter inside an image, or the like, by using a plurality of image frames resulting from the scan convert process. Also, the image processing circuitry 140 combines text information of various types of parameters, scale graduations, body marks, and the like with the ultrasound image data.

In other words, the F-mode data and the Doppler data are each ultrasound image data before the scan convert process. The data generated by the image processing circuitry 140 is the display-purpose ultrasound image data after the scan convert process. The E-mode data and the Doppler data may be referred to as raw data. From the two-dimensional ultrasound image data before the scan convert process, the image processing circuitry 140 is configured to generate display-purpose two-dimensional ultrasound image data.

Further, the image processing circuitry 140 is configured to generate three-dimensional B-mode image data by performing a coordinate transformation process on three-dimensional B-mode data generated by the B-mode processing circuitry 120. Further, the image processing circuitry 140 is configured to generate three-dimensional Doppler image data by performing a coordinate transformation process on three-dimensional Doppler data generated by the Doppler processing circuitry 130.

Further, the image processing circuitry 140 is configured to perform a rendering process on volume image data, to generate any of various types of two-dimensional image data for the purpose of displaying the volume image data on the display 103. Examples of the rendering process performed by the image processing circuitry 140 include a process of generating Multi Planar Reconstruction (MPR) image data from the volume image data, by implementing an MPR method. Further, examples of the rendering process performed by the image processing circuitry 140 also include a Volume Rendering (VR) process to generate two-dimensional image data reflecting information of a three-dimensional image. Further, examples of the rendering process performed by the image processing circuitry 140 also include a Surface Rendering (SR) process to generate two-dimensional image data obtained by extracting only surface information of a three-dimensional image.

The image processing circuitry 140 is configured to store the generated image data and the image data on which the various types of image processing processes have been performed, into the image memory 150. Additionally, together with the image data, the image processing circuitry 140 may also generate and store, into the image memory 150, information indicating a display position of each piece of image data, various types of information used for assisting operations on the ultrasound diagnosis apparatus 1, and additional information related to diagnosing processes such as patient information.

Further, the image processing circuitry 140 according to the first embodiment executes an image generating function 141, a schematic image obtaining function 142, an analyzing function 143, an image processing function 144, a display controlling function 145, and an estimating function 146. In this situation, the processing functions executed by the image generating function 141, the schematic image obtaining function 142, the analyzing function 143, the image processing function 144, the display controlling function 145, and the estimating function 146 are recorded in the storage circuitry 160 in the form of computer-executable programs, for example. The image processing circuitry 140 is a processor configured to realize the functions corresponding to the programs by reading and executing the programs from the storage circuitry 160. In other words, the image generating function 141 is a function realized as a result of the image processing circuitry 140 reading and executing a program corresponding to the image generating function 141 from the storage circuitry 160. The schematic image obtaining function 142 is a function realized as a result of the image processing circuitry 140 reading and executing a program corresponding to the schematic image obtaining function 142 from the storage circuitry 160. The analyzing function 143 is a function realized as a result of the image processing circuitry 140 reading and executing a program corresponding to the analyzing function 143 from the storage circuitry 160. The image processing function 144 is a function realized as a result of the image processing circuitry 140 reading and executing a program corresponding to the image processing function 144 from the storage circuitry 160. The display controlling function 145 is a function realized as a result of the image processing circuitry 140 reading and executing a program corresponding to the display controlling function 145 from the storage circuitry 160. The estimating function 146 is a function realized as a result of the image processing circuitry 140 reading and executing a program corresponding to the estimating function 146 from the storage circuitry 160. In other words, the image processing circuitry 140 that has read the programs has the functions indicated within the image processing circuitry 140 in FIG. 1. The functions of the image generating function 141, the schematic image obtaining function 142, the analyzing function 143, the image processing function 144, the display controlling function 145, and the estimating function 146 will be explained later.

With reference to FIG. 1, an example is explained in which the processing functions implemented by the image generating function 141, the schematic image obtaining function 142, the analyzing function 143, the image processing function 144, the display controlling function 145, and the estimating function 146 are realized by the single image processing circuit (i.e., the image processing circuitry 140). However, another arrangement is also acceptable in which processing circuitry is structured by combining together a plurality of independent processors, so that the functions are realized as a result of the processors executing the programs.

The term “processor” used in the above explanations denotes, for example, a Central Processing Unit (CPU), a Graphics Processing Unit (GPU), or a circuit such as an Application Specific integrated Circuit (ASIC) or a programmable logic device (e.g., a Simple Programmable Logic Device [SPLD], a Complex Programmable Logic Device [CPLD], or a Field Programmable Gate Array [FPGA]). The processors realize the functions by reading and executing the programs saved in the storage circuitry 160. In this situation, instead of saving the programs in the storage circuitry 160, it is also acceptable to directly incorporate the programs in the circuits of the processors. In that situation, the processors realize the functions by reading and executing the programs incorporated in the circuits thereof. The processors in the present embodiment do not each necessarily have to be structured as a single circuit. It is also acceptable to structure one processor by combining together a plurality of independent circuits so as to realize the functions thereof. Further, it is also acceptable to integrate two or more of the constituent elements in FIG. 1 into one processor so as to realize the functions thereof.

The image memory 150 is a memory configured to store therein, as the ultrasound image data, the image data such as the B-mode image data, the Doppler image data, or the like generated by the image processing circuitry 140. Further, the image memory 150 is also capable of storing therein, as the ultrasound image data, image data such as the B-mode data generated by the B-mode processing circuitry 120 or the Doppler data generated by the Doppler processing circuitry 130. After a diagnosis process, for example, the operator is able to invoke any of the ultrasound image data stored in the image memory 150. The invoked ultrasound image data can serve as display-purpose ultrasound image data after being routed through the image processing circuitry 140. Further, the image memory 150 is also capable of storing therein a schematic image 300 (see FIG. 6) that schematically indicates a part of the fetus, as two-dimensional bitmap image data (hereinafter “bitmap data”). Details of the schematic image 300 will be explained later.

The storage circuitry 160 is configured to store therein a control program for performing the ultrasound wave transmission/reception process, image processing processes, and display processes, diagnosis information (e.g., patients' IDs, observation of medical doctors) and various types of data such as diagnosis protocols, various types of body marks, and the like. Further, the storage circuitry 160 may also be used, as necessary, for storing therein any of the ultrasound image data and the bitmap data (the schematic image 300) stored in the image memory 150. Further, it is possible to transfer any of the data stored in the storage circuitry 160 to an external device via an interface unit (not illustrated).

The controlling circuitry 170 is configured to control the entirety of the processes performed by the ultrasound diagnosis apparatus 1. More specifically, the controlling circuitry 170 is configured to control processes of the transmission and reception circuitry 110, the B-mode processing circuitry 120, the Doppler processing circuitry 130, the image processing circuitry 140, and the like, on the basis of the various types of setting requests input by the operator via the input interface 102, and any of the various types of control programs and the various types of data read from the storage circuitry 160.

The transmission and reception circuitry 110, the B-mode processing circuitry 120, the Doppler processing circuitry 130, the image processing circuitry 140, the controlling circuitry 170, and the like built in the apparatus main body 100 may be configured by using hardware such as a processor (e.g., a Central Processing Unit [CPU], a Micro-Processing Unit [MPU], or an integrated circuit) or may be configured by using a program realized as modules in the form of software.

With the ultrasound diagnosis apparatus 1 structured as described above, for the purpose of, for example, checking the growth of the fetus in the uterus of the pregnant woman, the ultrasound probe 101 is configured to perform an ultrasound wave transmission/reception process (an ultrasound scan) on a region including a part of the fetus in the uterus of the pregnant woman, whereas the image processing circuitry 140 is configured to generate an ultrasound image rendering the region including the part of the fetus on the basis of a result of the scan. For example, by using the ultrasound image, the ultrasound diagnosis apparatus 1 is capable of measuring parameters such as the biparietal diameter (BPD), the head circumference (HC), the abdominal circumference (AC), the femur length (EL), the humerus length (HL), and the like of the fetus and is capable of calculating an estimated fetal weight (EFW), by using these parameters.

For example, as one of the parameters, the volume of predetermined range of a part (e.g., a thigh or an upper arm) of the fetus may be measured from the ultrasound image. The predetermined range is designated by an operation performed by the operator, for example. In this situation, to guide the operation performed by the operator, the ultrasound diagnosis apparatus 1 may display, on a display, an ultrasound image and the schematic image 300 schematically indicating a part of the fetus, so as to be kept in correspondence with each other.

However, the part of the fetus rendered in the ultrasound image may be displayed on the display in a different orientation from that of the part of the fetus indicated in the schematic image 300, in some situations. In those situations, when the operator looks at the ultrasound image and the schematic image on the display, the operator would feel strange.

To cope with these situations, when an ultrasound scan is performed on the region including a part of the fetus, the ultrasound diagnosis apparatus 1 according to the first embodiment is configured to generate an ultrasound image rendering the region including the part of the fetus, on the basis of a result of the ultrasound scan. Further, the ultrasound diagnosis apparatus 1 is configured to obtain the schematic image 300 schematically indicating the part of the fetus. For example, when the region on which the ultrasound scan is performed is a three-dimensional region, the ultrasound image is a three-dimensional image, and a tomographic image is generated from the three-dimensional image. Further, the ultrasound diagnosis apparatus 1 is configured to cause the display 103 to display the schematic image 300 and either the ultrasound image or the tomographic image, in such a manner that the orientation of the subject included in either the ultrasound image or the tomographic image and the orientation of the subject indicated in the schematic image 300 are close to each other, on the basis of a result of an analysis performed on either the ultrasound image or the image (the tomographic image) based on the ultrasound image. More specifically, on the basis of the result of the analysis performed on the tomographic image, the ultrasound diagnosis apparatus 1 is configured to perform at least one selected from between a rotating process and an inverting process on the schematic image 300 and to cause the display 103 to display the schematic image 300 resulting from the process, together with the image (the tomographic image) based on the ultrasound image.

With this arrangement, the ultrasound diagnosis apparatus 1 according to the first embodiment is able to cause the display 103 to display the part of the fetus rendered in the tomographic image in the same orientation as the orientation of the part of the fetus indicated in the schematic image 300 resulting from the process. It is therefore possible to reduce the strange feeling which the operator may experience while he/she is looking at the ultrasound image (the tomographic image) and the schematic image 300. Further, as one of the parameters explained above, the ultrasound diagnosis apparatus 1 is able to calculate (measure) the volume of the predetermined range of the part of the fetus from the ultrasound image (the tomographic image) and to calculate (estimate) an estimated fetal weight (EFW) by using the parameters. In this manner, by using the ultrasound diagnosis apparatus 1 according to the first embodiment, the operator is able to easily perform the measuring processes while using the ultrasound image (the tomographic image).

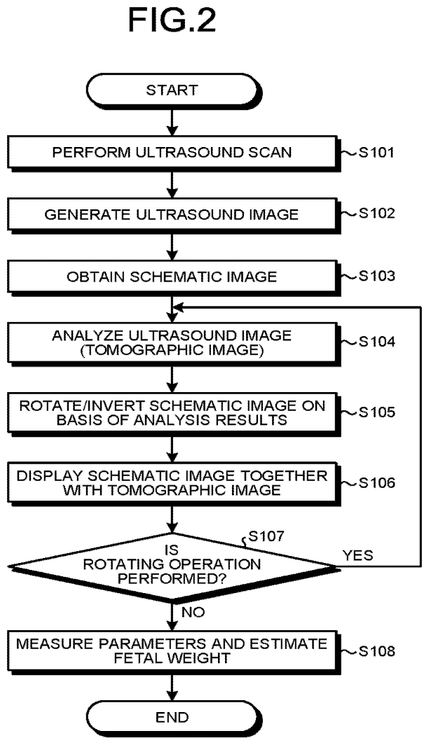

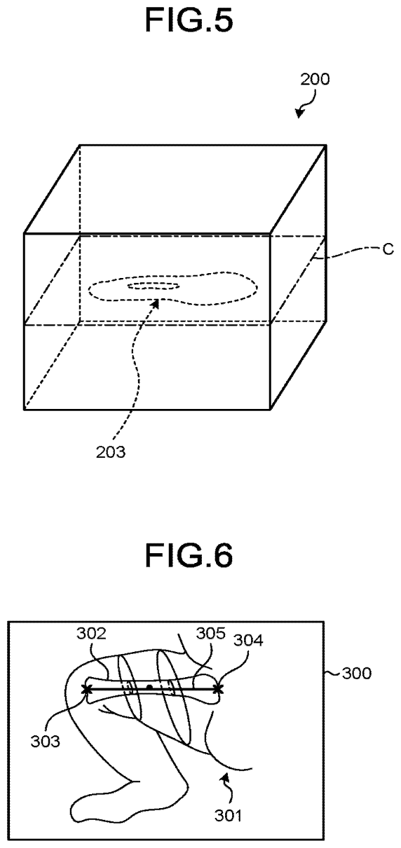

In the following sections, functions of the image generating function 141, the schematic image obtaining function 142, the analyzing function 143, the image processing function 144, and the display controlling function 145 that are executed by the image processing circuitry 140 will be explained, with reference to FIGS. 2 to 14.

FIG. 2 is a flowchart illustrating a procedure in a process performed by the ultrasound diagnosis apparatus 1 according to the first embodiment. FIG. 2 illustrates the flowchart explaining an operation (an image processing method) of the entirety of the ultrasound diagnosis apparatus 1, to explain which step in the flowchart each of the constituent elements corresponds.

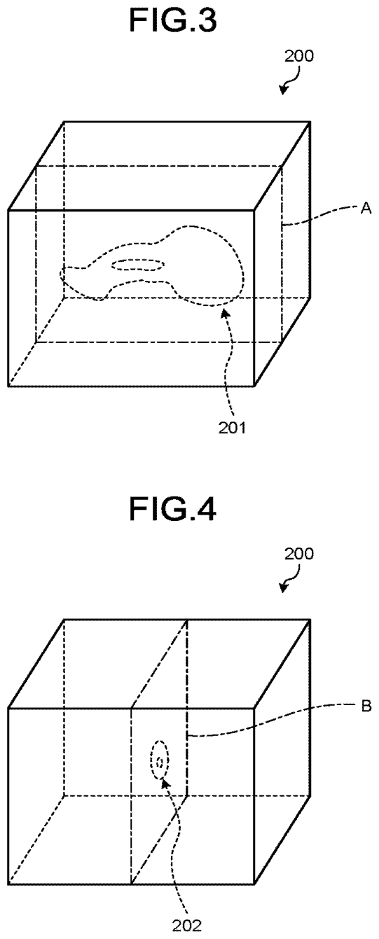

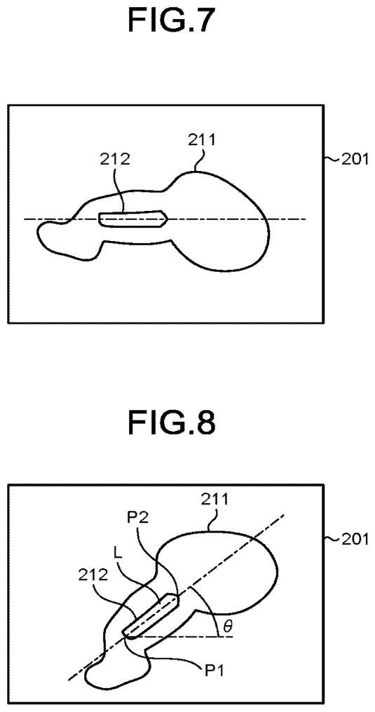

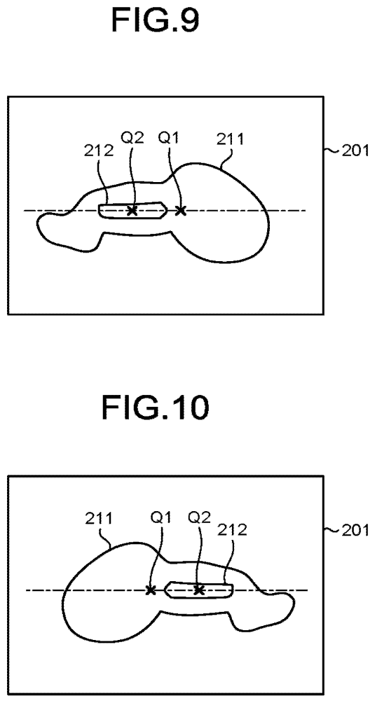

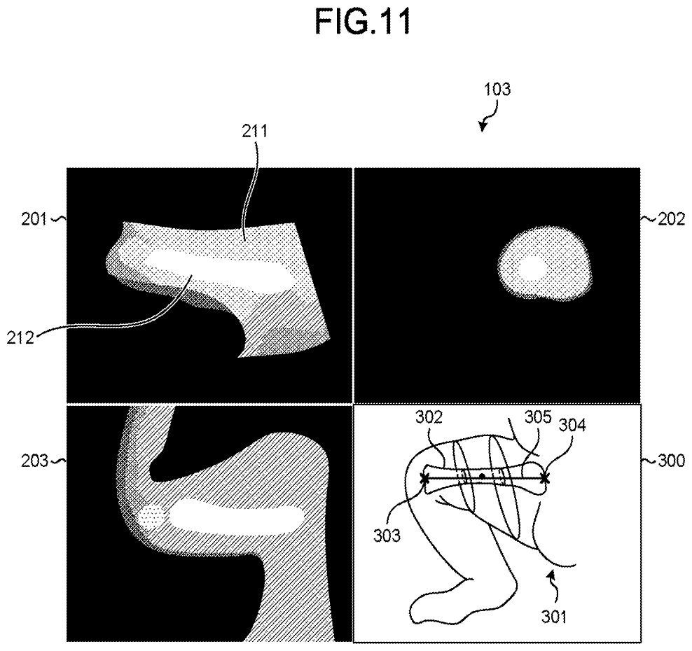

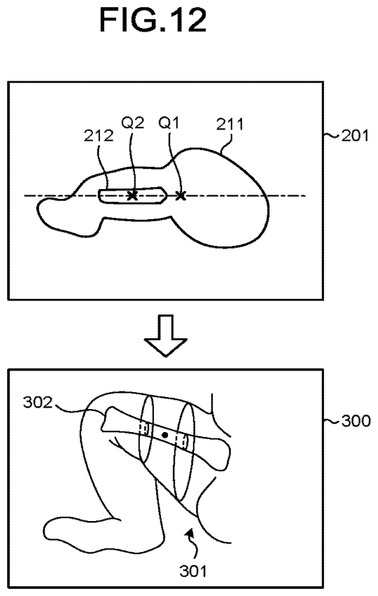

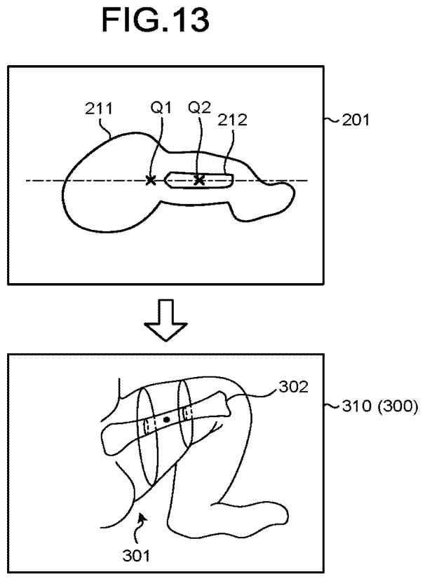

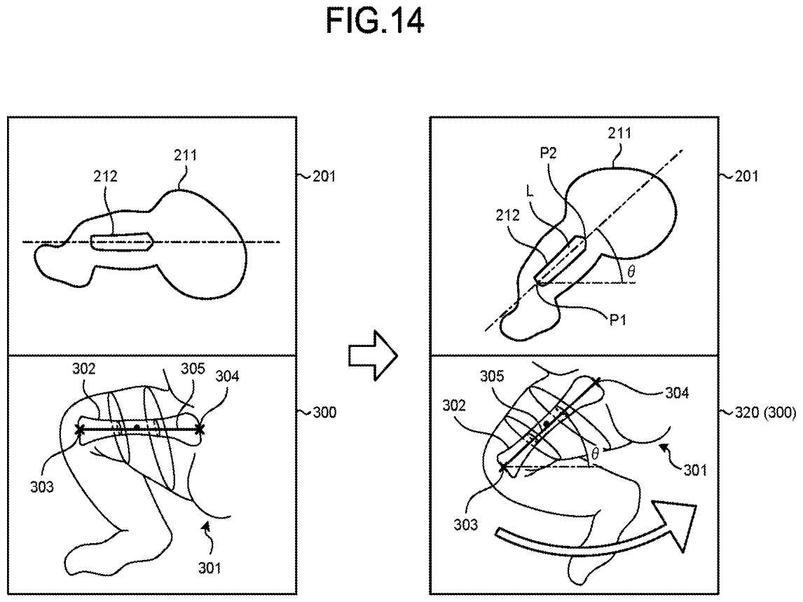

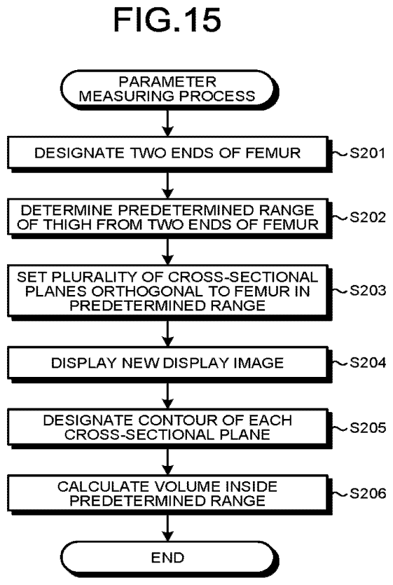

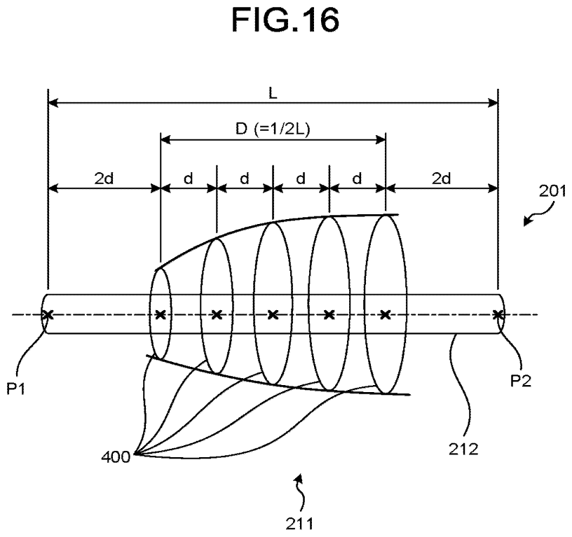

Further, in FIGS. 3 to 14, an example will be explained in which a thigh is used as a part of the fetus. FIGS. 3 to 5 are drawings for explaining examples of processes performed by the image generating function 141 of the ultrasound diagnosis apparatus 1 according to the first embodiment. FIG. 6 is a drawing for explaining an example of a process performed by the schematic image obtaining function 142 of the ultrasound diagnosis apparatus 1 according to the first embodiment. FIGS. 7 to 10 are drawings for explaining examples of processes performed by the analyzing function 143 of the ultrasound diagnosis apparatus 1 according to the first embodiment. FIGS. 11 to 14 are drawings for explaining examples of processes performed by the display controlling function 145 of the ultrasound diagnosis apparatus 1 according to the first embodiment.

Step S101 in FIG. 2 is a step performed by the ultrasound probe 101. At step S101, the ultrasound probe 101 is brought into contact with the body surface of the patient P (the abdomen of the pregnant women), performs an ultrasound scan on a region including a part (a thigh) of a fetus in the uterus of the pregnant woman, and acquires reflected-wave signals of the region as a result of the ultrasound scan. The ultrasound probe 101 is an example of a “scanning unit”.

Step S102 in FIG. 2 is a step performed as a result of the image processing circuitry 140 invoking the program corresponding to the image generating function 141 from the storage circuitry 160. At step S102, the image generating function 141 generates an ultrasound image rendering the region including the thigh, on the basis of the reflected-wave signals obtained by the ultrasound probe 101. In this situation, the image generating function 141 may generate the ultrasound image by generating B-mode image data while using the B-mode data generated by the B-mode processing circuitry 120 or may generate the ultrasound image by using the ultrasound image data stored in the image memory 150. The image generating function 141 is an example of a “generating unit”.

At step S102, the image generating function 141 generates an ultrasound image 200 illustrated in FIG. 3, for example. The ultrasound image 200 illustrated in FIG. 3 is a three-dimensional image (three-dimensional volume image data) rendering the region including the thigh of the fetus. From the ultrasound image 200, tomographic images 201 to 203 (FIGS. 3 to 5) are generated. The tomographic images 201, 202, and 203 are tomographic images taken on plane A, plane B, and plane C, respectively. In this situation, one tomographic image (a target tomographic image) selected from among the tomographic images 201 to 203 is used for designating the predetermined range of the thigh. In the present embodiment, an example will be explained in which the target tomographic image is the tomographic image 201.

Step S103 in FIG. 2 is a step performed as a result of the image processing circuitry 140 invoking the program corresponding to the schematic image obtaining function 142 from the forage circuitry 160. At step S103, the schematic image obtaining function 142 obtains the schematic image 300 stored in the image memory 150. The schematic image 300 is read from the image memory 150, when the operator is to perform a measuring process by using the ultrasound image 200 (the tomographic image 201). For this reason, instead of being performed after step S102, step S103 may be performed before step S101 or may be performed between step S101 and step S102. The schematic image obtaining function 142 is an example of an “obtaining unit”.

For example, the schematic image 300 is toyed in the image memory 150 while being kept in correspondence with measured items. Examples of the measured items include the “head (fetal head)”, the “abdomen”, a “thigh”, an “upper arm”, of the fetus. For example, when measuring a thigh of the fetus, the operator selects “thigh” as a measured item. In that situation, at step S103, the schematic image obtaining function 142 obtains the schematic image 300 kept in correspondence with the measured item “thigh” from the image memory 150.

As illustrated in FIG. 6, the schematic images 300 obtained by the schematic image obtaining function 142 schematically indicates, for example, the right leg of the fetus including the thigh and includes: a thigh image region 301 that is an image region indicating the exterior shape of the thigh of the fetus; and a femur image region 302 that is an image region indicating the exterior shape of the bone (the femur) in the thigh. In this situation, to guide operations performed by the operator, the schematic image 300 illustrated in FIG. 6 may further include points 303 and 304 indicating the two ends of the femur of the fetus and a line 305 connecting the two ends (the points 303 and 304) to each other. Examples of the operations performed by the operator include an operation performed by the operator to designate the two ends of the femur from the tomographic image 201 while using the input interface 102, during the process (a parameter measuring process) of measuring the volume of the predetermined range of the thigh. As explained herein, the schematic image 300 is an image including information related to the measuring method (the parameter measuring process) implemented on the part (the thigh in the present example) of the fetus. The parameter measuring process will be explained later.

Step S104 in FIG. 2 is a step performed as a result of the image processing circuitry 140 invoking the program corresponding to the analyzing function 143 from the storage circuitry 160. At step S104, the analyzing function 143 analyzes the tomographic image 201 that is a target tomographic image of the ultrasound image 200 obtained at step S102. The analyzing function 143 is an example of an “analyzing unit”.

At step S104, as illustrated in FIG. 7, for example, the analyzing function 143 detects: a thigh image region 211 that is an image region indicating the exterior shape of the thigh rendered in the tomographic image 201; and a femur image region 212 that is an image region indicating the exterior shape of the bone (the femur) in the thigh. Possible methods for detecting the thigh image region 211 and the femur image region 212 include a first method and a second method described below.

In the first method, at first, the analyzing function 143 calculates a histogram of an image of the region of the entire tissue or inside a Region of Interest (ROI) within the tomographic image 201 and sets threshold values for detecting the thigh image region 211 and he femur image region 212 with the histogram, as a first threshold value and a second threshold value. Subsequently, the analyzing function 143 binarizes the image by using the first and the second threshold values. For example, by eliminating noise while using a morphology calculation or the like, the analyzing function 143 detects the thigh image region 211 and the femur image region 212 from the tomographic image 201.

In the second method, at first, a plurality of pieces of data are prepared in each of which a known tomographic image is kept in correspondence with a thigh image region and a femur image region. The analyzing function 143 learns the thigh image regions and the femur image regions from the plurality of pieces of data by using a Convolutional Neural Network (CNN). In this situation, because the algorithm of the CNN or the like is empirically learned and because the fetus grows in the uterus, the data used in the learning process does not have to be data from the same fetus. Subsequently, on the basis of the learning, the analyzing function 143 detects the thigh image region 211 and the femur image region 212 from the tomographic image 201.

The analyzing function 143 generates this detection result as an analysis result. In other words, analysis results include information indicating the thigh image region 211 and the femur image region 212 in the tomographic image 201.

Further, at step S104, for example, the analyzing function 143 detects the orientation of the femur from the femur image region 212 in the tomographic image 201. As a method for detecting the orientation of the femur, the method described below may be used. This method can use the same algorithm as the one used for measuring the femur length (FL).

At first, as illustrated in FIG. 8, within the femur image region 212 in the tomographic image 201, the analyzing function 143 searches for points P1 and P2 indicating the two ends of the femur and a line L connecting the two ends (the points P1 and P2) to each other. After that, the analyzing function 143 detects an angle θ of the line L as the orientation of the femur, by calculating a bounding rectangle while using a rotating calipers method or the like, for example. For instance, the detected orientation of the femur indicates that, when the width direction of the image is used as a reference, the femur is tilted counterclockwise by the angle θ.

The analyzing function 143 also generates this detection result as an analysis result. In other words, the analysis results further include information indicating the orientation of the femur in the femur image region 212 in the tomographic image 201.

Further, at step S104, for example, the analyzing function 143 detects a positional relationship between the thigh image region 211 and the femur image region 212 in the tomographic image 201. As a method for detecting the positional relationship between the thigh image region 211 and the femur image region 212, the method described below may be used.

At first, as illustrated in FIGS. 9 and 10, the analyzing function 143 searches for a center of gravity Q1 of the thigh in the thigh image region 211 and searches for center of gravity Q2 of the femur in the femur image region 212. For example, as illustrated in FIG. 9, when the center of gravity Q1 is positioned on the right-hand side of the center of gravity Q2, the positional relationship between the thigh image region 211 and the femur image region 212 is detected as a first positional relationship. In another example, as illustrated in FIG. 10, when the center of gravity Q1 is positioned on the left-hand side of the center of gravity Q2, the positional relationship between the thigh image region 211 and the femur image region 212 is detected as a second positional relationship.

The analyzing function 143 also generates this detection result as an analysis result. In other words, the analysis results further include information indicating the positional relationship (the first or the second positional relationship) between the thigh image region 211 and the femur image region 212 in the tomographic image 201.

Step S105 in FIG. 2 is a step performed as a result of the image processing circuitry 140 invoking the program corresponding to the image processing function 144 from the storage circuitry 160. At step S105, the image processing function 144 performs at least one selected from between a rotating process and an inverting process on the schematic image 300 obtained at step S103, on the basis of the analysis results (the thigh image region 211 and the femur image region 212 in the tomographic image 201, the orientation of the femur, and the positional relationship between the thigh image region 211 and the femur image region 212) obtained at step S104. The image processing function 144 is an example of a “processing unit”.

Step S106 in FIG. 2 is a step performed as a result of the image processing circuitry 140 invoking the program corresponding to the display controlling function 145 from the storage circuitry 160. At step S106, as illustrated in FIG. 11, the display controlling function 145 causes the display 103 to display the tomographic images 201 to 203 of the ultrasound image 200 obtained at step S102 and the schematic image 300 resulting from the abovementioned process performed at step S105. One tomographic image (the target tomographic image) selected from among the tomographic images 201 to 203 is used for designating the predetermined range of the thigh. Accordingly, the display controlling function 145 does not necessarily have to cause the display 103 to display all the tomographic images 201 to 203. The display controlling function 145 may cause the display 103 to display the tomographic image 201 serving as the target tomographic image and the schematic image 300 resulting from the abovementioned process. The display controlling function 145 is an example of a “display controlling unit”.

Next, a specific example will be explained in which, as a result of the processes at steps S105 and S106, the thigh rendered in the tomographic image 201 is displayed on the display 103 in the same orientation as the orientation of the thigh indicated in the schematic image 300.

For example, as illustrated in FIG. 12, in the analysis results, the positional relationship between the thigh image region 211 and the femur image region 212 is indicated as the first positional relationship. In other words, the center of gravity Q1 of the thigh in the thigh image region 211 is positioned on the right-hand side of the center of gravity Q2 of the femur in the femur image region 212. In that situation, the image processing function 144 does not invert the schematic image 300 obtained at step S103, so that the display controlling function 145 causes the display 103 to display the schematic image 300 as is. For example, in FIG. 12, the display 103 displays the schematic image 300 schematically indicating the right leg of the fetus including the thigh.

In another example, as illustrated in FIG. 13, in the analysis results, the positional relationship between the thigh image region 211 and the femur image region 212 is indicated as the second positional relationship. In other words, the center of gravity Q1 of the thigh in the thigh image region 211 is positioned on the left-hand side of the center of gravity Q2 of the femur in the femur image region 212. In that situation, the image processing function 144 inverts he schematic image 300 obtained at step S103, so that the display controlling function 145 causes the display 103 to display the schematic image 300 resulting from the inverting process. For example, in FIG. 13, the display 103 displays a schematic image 310 schematically indicating the left leg of the fetus including the thigh, as the schematic image 300 resulting from the inverting process.

In yet another example, as illustrated in FIG. 14, in the analysis results, the orientation of the femur is indicated as being tilted counterclockwise by the angle θ, when the width direction of the image is used as a reference. In that situation, the image processing function 144 rotates the schematic image 300 obtained at step S103 counterclockwise by the angle θ, so that the display controlling function 145 causes the display 103 to display the schematic image 300 resulting from the rotating process. For example, in FIG. 14, the display 103 displays a schematic image 320 schematically indicating the right leg of the fetus including the thigh and having been rotated by the angle θ, as the schematic image 300 resulting from the rotating process.

In yet another example, in the analysis results, it is indicated that the positional relationship between the thigh image region 211 and the femur image region 212 is the second positional relationship and that the orientation of the femur is tilted counterclockwise by the angle θ when the width direction of the image is used as a reference. In that situation, the image processing function 144 inverts the schematic image 300 obtained at step S103 and rotates the inverted result counterclockwise by the angle θ, so that the display controlling function 145 causes the display 103 to display the schematic image 300 resulting from the inverting and the rotating processes.

In the manner described above, steps S101 through S106 are performed in a real-time manner. In other words, every time an ultrasound image 200 is generated, the image processing function 144 performs at least one selected from between a rotating process and an inverting process on the schematic image 300 on the basis of the analysis results from the analysis performed on either the ultrasound image 200 or the image (the tomographic image 201) based on the ultrasound image 200. Every time at least one of the processes is performed, the display controlling function 145 causes the display 103 to display the schematic image 300 resulting from the process and either the ultrasound image 200 or the tomographic image 201.

Step S107 in FIG. 2 is a step performed by the input interface 102, while the tomographic image 201 and the schematic image 300 are displayed on the display 103. At step S107, by using the input interface 102, the operator performs operations to enlarge or reduce the size, to rotate, and/or to move the tomographic image 201 serving as the target tomographic image. For example, when the operator performs a rotating operation to rotate the tomographic image 201 by using the input interface 102 (step S107: Yes), the processes at steps S104 through S106 explained above are performed again. In that situation, at step S104, the analyzing function 143 generates analysis results explained above; at step S105, the image processing function 144 rotates the schematic image 300; and at step S106, the display controlling function 145 causes the display 103 to display the schematic image 300 resulting from the rotating process.

In contrast, when no operation such as the rotating operation described above or the like is performed within a predetermined period of time (step S107: No), the process at step S108 explained below will be performed.

Step S108 in FIG. 2 is a step performed as a result of the image processing circuitry 140 invoking the program corresponding to the estimating function 146 from the storage circuitry 160. As explained above, by using the ultrasound image, the ultrasound diagnosis apparatus 1 is capable of measuring the parameter indicating the volume of the predetermined range of the thigh from the tomographic image 201 of the fetus, in addition to the parameters such as the biparietal diameter (BPD), the head circumference (HC), the abdominal circumference (AC), the femur length (FL), the humerus length (HL), and the like of the fetus. For example, by performing the parameter measuring process (FIG. 15) explained below, the estimating function 146 is configured to calculate (measure) the volume of the predetermined range of the thigh from the tomographic image 201, as one of the parameters. After that, by using the parameters, the estimating function 146 is configured to calculate (estimate) the estimated fetal weight (EFW).

Next, the process of measuring the volume of the predetermined range of the thigh will specifically be explained as a part (the parameter measuring process) of the process at step S108. FIG. 15 is a flowchart illustrating a procedure in the parameter measuring process performed by the ultrasound diagnosis apparatus 1 according to the first embodiment. FIG. 16 is a drawing for explaining an example of a process performed by the estimating function 146 of the ultrasound diagnosis apparatus 1 according to the first embodiment.

At step S201 in FIG. 15, at first, the two ends of the femur rendered in the tomographic image 201 are designated. For example, as illustrated in FIG. 16, in the femur image region 212 of the tomographic image 201, the points P1 and P2 indicating the two ends of the femur are designated. The points P1 and P2 are designated by the estimating function 146. Alternatively, the operator may designate the points P1 and P2 by operating the input interface 102.

At step S202 in FIG. 15, when the points P1 and P2 indicating the two ends of the femur rendered in the tomographic image 201 have been designated, the estimating function 146 determines a predetermined range of the thigh rendered in the tomographic image 201. For example, as illustrated in FIG. 16, when a line connecting the two ends (the points P1 and P2) of the femur to each other in the tomographic image 201 is expressed as L, a predetermined range D corresponds to a central part of the thigh image region 211 in the tomographic image 201, while the length thereof is set to a half of the distance between the two ends of the femur (i.e., ½L).

At step S203 in FIG. 15, in the thigh image region 211, the estimating function 146 sets a plurality of cross-sectional planes 400 that are orthogonal to the femur in the predetermined range D, at regular intervals d. For example, as illustrated in FIG. 16, when d=D/4 is satisfied, the number of cross-sectional planes 400 in the predetermined range D is five.

At step S204 in FIG. 15, the display controlling function 145 causes the display 103 to display the plurality of cross-sectional planes 400. As a method for displaying the cross-sectional planes 400, the display controlling function 145 may cause the display 103 to display a new display image including the plurality of cross-sectional planes 400 in the predetermined range D in the thigh image region 211 and the femur image region 212 in which the two ends (the points P1 and P2) of the femur are designated, together with the tomographic images 201 to 203 and the schematic image 300. Alternatively, the display controlling function 145 may cause the display 103 to display the abovementioned display image, separately from the tomographic images 201 to 203 and the schematic image 300.

At step S205 in FIG. 15, the contour of each of the plurality of cross-sectional planes 400 is designated. For example, as illustrated in FIG. 16, the contour of each of the cross-sectional planes 400 is designated by the estimating function 146 while using the brightness levels of the tomographic images 201 to 203. Alternatively, the contour of each of the cross-sectional planes 400 may be designated by the operator by drawing with the use of the input interface 102.

At step S206 in FIG. 15, the estimating function 146 calculates a volume Vol of the inside of the predetermined range D of the thigh rendered in the tomographic image 201, by using the contours and the intervals d of the cross-sectional planes 400. In this situation, the volume Vol can be expressed by Mathematical Formula 1.

$$Vol = \frac{1}{2} {\sum }_{i = 1}^{N - 1} \{ ( {S}_{i } + {S}_{i + 1} ) \cdot d \} \qquad ( 1 )$$

In Mathematical Formula 1, Si denotes the area of an i-th cross-sectional plane 400, where i is an integer from 1 to (N-1). The letter “N” denotes the number of cross-sectional planes 400 and is “5” in the example illustrated in FIG. 16. Further, by using the calculated volume Vol as a parameter, the estimating function 146 calculates (estimates) the estimated fetal weight (EFW).

As explained above, when the ultrasound diagnosis apparatus 1 according to the first embodiment is used, when the ultrasound scan is performed on the region including a part (the thigh) of the fetus, the image generating function 141 is configured to generate the ultrasound image 200 rendering the region including the thigh on the basis of a result of the ultrasound scan. The schematic image obtaining function 142 is configured to obtain the schematic image 300 schematically indicating the thigh. In this situation, when the region on which the ultrasound scan is performed is a three-dimensional region, the ultrasound image 200 is a three-dimensional image, so that the tomographic image 201 is generated from the three-dimensional image. Further, the image processing function 144 performs at least one selected from between a rotating process and an inverting process on the schematic image 300, on the basis of the analysis results from the analysis performed on the ultrasound image 200 (the tomographic image 201). The display controlling function 145 causes the display 103 to display the schematic image 300 resulting from the process, together with the image (the tomographic image 201) based on the ultrasound image 200. With these arrangements, the ultrasound diagnosis apparatus 1 according to the first embodiment is configured to cause the display 103 to display the thigh rendered in the ultrasound image 200 (the tomographic image 201) in the same orientation as the orientation of the thigh indicated in the schematic image 300 resulting from the process. It is therefore possible to reduce the strange feeling which the operator may experience while looking at the ultrasound image 200 (the tomographic image 201) and the schematic image 300. As a result, by using the ultrasound diagnosis apparatus 1 according to the first embodiment, the operator is able to easily perform the measuring processes using the ultrasound image 200 (the tomographic image 201).

Further, when the ultrasound diagnosis apparatus 1 according to the first embodiment is used, the analyzing function 143 is configured to analyze the ultrasound image 200 (the tomographic image 201), so that the image processing function 144 is configured to perform at least one selected from between a rotating process and an inverting process on the schematic image 300, on the basis of the analysis results obtained by the analyzing function 143. For example, the analyzing function 143 analyzes the orientation of the bone (the femur) included in the part (the thigh) of the fetus from the ultrasound image 200 (the tomographic image 201). The orientation of the femur is one of the analysis results obtained by the analyzing function 143. On the basis of the orientation of the femur, the image processing function 144 is configured to rotate the schematic image 300. Further, the display controlling function 145 is configured to cause the display 103 to display the schematic image 300 resulting from the rotating process, together with the image (the tomographic image 201) based on the ultrasound image 200. With these arrangements, the ultrasound diagnosis apparatus 1 according to the first embodiment is configured to cause the display 103 to display the thigh rendered in the ultrasound image 200 (the tomographic image 201) in the same orientation as the orientation of the thigh indicated in the schematic image 300 resulting from the rotating process. It is therefore possible to reduce the strange feeling which the operator may experience while looking at the ultrasound image 200 (the tomographic image 201) and the schematic image 300.

Further, when the ultrasound diagnosis apparatus 1 according to the first embodiment is used, as an analysis performed on the ultrasound image 200 (the tomographic image 201), the analyzing function 143 is configured to analyze the positional relationship between the image region (the thigh image region 211) indicating the part (the thigh) of the fetus and the bone image region (the femur image region 212) indicating the bone (the femur) included in the thigh, from the ultrasound image 200 (the tomographic image 201). More specifically, the analyzing function 143 analyzes the positional relationship between the center of gravity of the thigh indicated in the thigh image region 211 and the center of gravity of the femur indicated in the femur image region 212. The positional relationship is one of the analysis results obtained by the analyzing function 143. On the basis of the positional relationship, the image processing function 144 is configured to invert the schematic image 300. Further, the display controlling function 145 is configured to cause the display 103 to display the schematic image 300 resulting from the inverting process, together with the image (the tomographic image 201) based on the ultrasound image 200. With these arrangements, the ultrasound diagnosis apparatus 1 according to the first embodiment is configured to cause the display 103 to display the thigh rendered in the ultrasound image 200 (the tomographic image 201) in the same orientation as the orientation of the thigh indicated in the schematic image 300 resulting from the inverting process. It is therefore possible to reduce the strange feeling which the operator may experience while looking at the ultrasound image 200 (the tomographic image 201) and the schematic image 300.

When the ultrasound diagnosis apparatus 1 according to the first embodiment is used, when the region on which an ultrasound scan is performed is a two-dimensional region, the ultrasound image 200 is the tomographic image 201. In that situation, the display controlling function 145 is configured to cause the display 103 to display the schematic image 300 resulting from at least one selected from between a rotating process and an inverting process, together with the ultrasound image 200 (the tomographic image 201). In this manner, even when the region on which the ultrasound scan is performed is a two-dimensional region, the ultrasound diagnosis apparatus 1 according to the first embodiment is able to reduce the strange feeling which the operator may experience while looking at the ultrasound image 20C (the tomographic image 201) and the schematic image 300. Further, in the first embodiment above, the example is explained in which a part of the fetus is a thigh. However, possible embodiments are not limited to this example. For example, the first embodiment described above is applicable to the situation where a part of the fetus is an upper arm.

Further, in the first embodiment described above, another arrangement is also acceptable in which the operator is able to switch between the situation where a part of the fetus is a thigh and the situation where a part of the fetus is an upper arm, by operating the input interface 102, so as to calculate the volume Vol of the inside of the predetermined range D for the thigh and for the upper arm.

Further, in the first embodiment described above, the analyzing function 143 is configured to detect the bone image region (e.g., the femur image region 212) as a bone (e.g., the femur) included in a part (e.g., the thigh) of the fetus, from the ultrasound image 200 (the tomographic image 201) and is configured to detect the orientation of the bone from the bone image region. However, it is not necessarily always possible to accurately detect the orientation of the bone. For example, there may be situations where the bone is not rendered clearly in the tomographic image 201 or where the bone is rendered only partially. In those situations, when the image processing function 144 rotates the schematic image 300 on the basis of an inaccurately detected orientation of the bone, there is a possibility that the operator may feel strange while he/she is looking at the ultrasound image 200 (the tomographic image 201) and the schematic image 300.

To cope with this problem, it is also acceptable to perform the processes as follows: At step S104 in FIG. 2, when having detected the bone image region (e.g., the femur image region 212) as a bone (e.g., the femur) included in a part (e.g., the thigh) of the fetus from the ultrasound image 200 (the tomographic image 201), the analyzing function 143 calculates a reliability of the detected bone image region. At step S105 in FIG. 2, when the reliability calculated by the analyzing function 143 is higher than a threshold value, the image processing function 144 performs at least one selected from between a rotating process and an inverting process on the schematic image 300, on the basis of the analysis results obtained by the analyzing function 143.

Examples of the reliability calculated at step S104 by the analyzing function 143 include: a reliability (hereinafter, “reliability Ra”) of the aspect ratio of the bone image region; a reliability (hereinafter, “reliability Rb”) of the ratio of the bone image region to a screen size (the tomographic image 201); and a reliability (hereinafter, “reliability Rc”) of a variance of a distribution of brightness levels in the bone image region. In this situation, when the reliabilities Ra, Rb, and Rc are applied to a serial model, an overall reliability R can be expressed as R=Ra×Rb×Rc.

For example, when the reliability Ra, Rb, and Rc are each “0.9”, the overall reliability R is equal to “0.729”. In this situation, when the threshold value is “0.7”, the reliability R “0.729” is higher than the threshold value “0.7”. Accordingly, at step S105, the image processing function 144 performs at least one selected from between a rotating process and an inverting process on the schematic image 300, on the basis of the analysis results obtained by the analyzing function 143. After that, at step S106, the display controlling function 145 causes the display 103 to display the schematic image 300 resulting from the process, together with the ultrasound image 200 (the tomographic image 201). At this time, the display controlling function 145 may cause the display 103 to display the reliability R “0.729” as a reliability of the ultrasound image 200 (the tomographic image 201) or may cause the display 103 to display information indicating that the reliability R is higher an the threshold value.

On the contrary, when the reliability Ra, Rb, and Rc are “0.9”, “0.8”, and “0.8”, respectively, an overall reliability R is equal to “0.576”. In this situation, the reliability R “0.576” is no higher than the threshold value “0.7”. In that situation, at step S105, the image processing function 144 does not perform either of the rotating and the inverting processes on the schematic image 300. At step S106, the display controlling function 145 causes the display 103 to display the schematic image 300 on which neither of the processes has been performed, together with the ultrasound image 200 (the tomographic image 201). At this time, the display controlling function 145 may cause the display 103 to display the reliability R “0.576” as a reliability of the ultrasound image 200 (the tomographic image 201) or may cause the display 103 to display information indicating that the reliability R is no higher than the threshold value.

### Second Embodiment

An overall configuration of the ultrasound diagnosis apparatus 1 according to a second embodiment is the same as the configuration illustrated in FIG. 1. Accordingly, in the second embodiment, some of the explanations that are duplicate of those in the first embodiment will be omitted.

With the ultrasound diagnosis apparatus 1 according to the first embodiment, the example was explained in which the schematic image 300 is represented by the bitmap data. However, according to the image display method using the bitmap data, the display 103 displays the schematic image 300 as an array of points called dots (which hereinafter will be referred to as a “dot array”). For this reason, every time the display controlling function 145 causes the display 103 to display the schematic image 300 resulting from at least one selected from between a rotating process and an inverting process, the display controlling function 145 needs to perform the process of changing the dot array.

To cope with this situation, with the ultrasound diagnosis apparatus 1 according to the second embodiment, the schematic image 300 may be represented by vector data. For example, in the second embodiment, the schematic image 300 stored in the image memory 150 may be converted from the bitmap data to the vector data in advance. According to an image display method using the vector data, the display 103 displays the schematic image 300 after a calculating process is performed based on numerical value data such as coordinates of points and lines (vectors) connecting the points, or the like. Accordingly, it is sufficient when the display controlling function 145 performs a coordinate transformation process when causing the display 103 to display the schematic image 300 resulting from at least one selected from between a rotating process and an inverting process. Consequently, the ultrasound diagnosis apparatus 1 according to the second embodiment is able to reduce the load of processing performed by the processor, in comparison to that in the first embodiment.

Further, with the ultrasound diagnosis apparatus 1 according to the second embodiment, because the schematic image 300 is represented by the vector data, another advantageous effect is also achieved where the image quality is not degraded. For example, when the operator performs an operation to enlarge or reduce the tomographic image 201 by using the input interface 102, the image processing function 144 enlarges or reduces the schematic image 300 in accordance with the operation, so that the display controlling function 145 causes the display 103 to display the schematic image 300 resulting from the enlarging or reducing process. When the schematic image 500 is represented by the bitmap data, the image quality is degraded by the enlarging/reducing process. In contrast, when the schematic image 300 is represented by the vector data, the image quality is not degraded by the enlarging/reducing process.

### Other Embodiments

It is possible carry out the present disclosure in various different forms other than those explained in the above embodiments.

In the above embodiments, the example is explained in which the ultrasound image 200 rendering the region including a part of the fetus is used as an ultrasound image rendering a region including a part of a subject. However, possible examples of ultrasound images to which the image processing methods explained in the above embodiments can be applied are not limited to this example. For instance, the image processing methods according to the present embodiments are similarly applicable to a situation where the ultrasound image 200 is an image rendering an organ such as the heart as a region including a part of a subject, so that the organ is measured by using the image.

Further, in the above embodiments, the display controlling function 145 causes the display 103 to display the schematic image 300 and either the ultrasound image 200 or the tomographic image 201, in such a manner that the orientation of the subject included in either the ultrasound image 200 or the tomographic image 201 and the orientation of the subject indicated in the schematic image 300 are close to each other, on the basis of the analysis results from the analysis performed on either the ultrasound image 200 or the image (tomographic image 201) based on the ultrasound image 200. More specifically, at step S105, the image processing function 144 performs at least one selected from between rotating process and an inverting process on the schematic image 300 on the basis of the analysis results. At step S106, the display controlling function 145 causes the display 103 to display the schematic image 300 resulting from the process and either the ultrasound image 200 or the tomographic image 201. However, possible embodiments are not limited to this example.

In a modification example of the above embodiments, for instance, at step S105, the image processing function 144 may perform at least one selected from between a rotating process and an inverting process on either the ultrasound image 200 or the tomographic image 201, on the basis of the analysis results. In that situation, at step S106, the display controlling function 145 causes the display 103 to display the image (either the ultrasound image 200 or the tomographic image 201) resulting from the process and the schematic image 300.

In that situation also, the processes at steps S101 through S106 described above are performed in a real-time manner. In other words, every time an ultrasound image 200 is generated, the image processing function 144 performs at least one selected from between a rotating process and an inverting process on either the ultrasound image 200 or the tomographic image 201, on the basis of the analysis results from the analysis performed on either the ultrasound image 200 or the image (the tomographic image 201) based on the ultrasound image 200. Every time at least one of the processes is performed, the display controlling function 145 causes the display 103 to display the image (either the ultrasound image 200 or the tomographic image 201) resulting from the process and the schematic image 300.

Further, in another modification example of the above embodiments, the image processing function 144 does not necessarily have to perform either of the rotating and inverting processes on the image. For example, the image memory 150 may store therein a plurality of schematic images 300 taken at mutually-different angles so that at step S105, the image processing function 144 searches for a schematic image 300 rendering an orientation close to the orientation of the subject included in either the ultrasound image 200 or the tomographic image 201, from among the plurality of schematic images 300 stored in the image memory 150. In that situation, at step S106, the display controlling function 145 causes the display 103 to display the schematic image 300 found in the search, together with either the ultrasound image 200 or the tomographic image 201.

More specifically, the image memory 150 stores therein a plurality of schematic images 300 exhibiting the first positional relationship and a plurality of schematic images 300 exhibiting the second positional relationship. For example, when a part of the subject represents a thigh of the fetus, as explained above, the first positional relationship denotes that the center of gravity Q1 of the thigh is positioned on the right-hand side of the center of gravity Q2 of the femur (see FIG. 9), whereas the second positional relationship denotes that the center of gravity Q1 of the thigh is positioned on the left-hand side of the center of gravity Q2 of the femur (see FIG. 10). For example, the plurality of schematic images 300 exhibiting the first positional relationship are obtained by rotating a schematic image 300 exhibiting the first positional relationship and being used as a reference, by one degree at a time from −90 degrees to 90 degrees. For example, the plurality of schematic images 300 exhibiting the second positional relationship are obtained by rotating a schematic image 300 exhibiting the second positional relationship and being used as a reference, by one degree at a time from −90 degrees to 90 degrees.

For instance, let us discuss an example in which the obtained analysis results indicate that the positional relationship between the thigh image region 211 and the femur image region 212 in the tomographic image 201 is the first positional relationship (see FIG. 12), while the orientation of the femur is tilted counterclockwise by the angle θ when the width direction of the image is used as a reference (FIG. 14). In this situation, the image processing function 144 selects a schematic image 300 in which the orientation of the femur is tilted counterclockwise by the angle θ, from among the plurality of schematic images 300 exhibiting the first positional relationship and being stored in the image memory 150. When there is no schematic image 300 in which the femur is tilted counterclockwise by the angle θ, the image processing function 144 selects one of the schematic images 300 in which the orientation of the femur is tilted counterclockwise at an angle closest to the angle θ. Further, the display controlling function 145 causes the display 103 to display the selected schematic image 300 and either the ultrasound image 200 or the tomographic image 201.

Similarly, for instance, let us discuss another example in which the obtained analysis results indicate that the positional relationship between the thigh image region 211 and the femur image region 212 in the tomographic image 201 is the second positional relationship (see FIG. 13), while the orientation of the femur is tilted clockwise by the angle θ when the width direction of the image is used as a reference. In this situation, the image processing function 144 selects a schematic image 300 in which the orientation of the femur is tilted clockwise by the angle θ, from among the plurality of schematic images 300 exhibiting the second positional relationship and being stored in the image memory 150. When there is no schematic image 300 in which the femur is tilted clockwise by the angle θ, the image processing function 144 selects one of the schematic images 300 in which the orientation of the femur is tilted clockwise at an angle closest to the angle θ. Further, the display controlling function 145 causes the display 103 to display the selected schematic image 300 and either the ultrasound image 200 or the tomographic image 201.

In the above embodiments, the example is explained in which, at step S104, the analyzing function 143 analyzes the orientation of the bone included in the part of the subject from either the ultrasound image 200 or the image (the tomographic image 201) based on the ultrasound image 200, so that at step S105, the image processing function 144 rotates the schematic image 300 on the basis of the orientation of the bone; however, possible embodiments are not limited to this example. Another arrangement is also acceptable in which, at step S104, the analyzing function 143 analyzes the orientation of a structure included in a part of the subject, from either the ultrasound image 200 or the image (tomographic image 201) based on the ultrasound image 200 so that, at step S105, the image processing function 144 rotates the schematic image 300 on the basis of the orientation of the structure. In this situation, examples of the structure include a valve of the heart, a blood vessel, and the like.

Further, possible embodiments are not limited to the embodiments described above. For instance, the image processing circuitry 140 may be a workstation provided separately from the ultrasound diagnosis apparatus 1. In that situation, the workstation includes processing circuitry that is the same as the image processing circuitry 140, so as to perform the processes described above.

Further, the constituent elements of the apparatuses and the devices illustrated in the drawings of the embodiments are based on functional concepts. Thus, it is not necessary to physically configure the constituent elements as indicated in the drawings. In other words, specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analyzed and executed by the CPU or may be realized as hardware using wired logic.

Further, the image processing methods explained in the above embodiments may be realized by causing a computer such as a personal computer or a workstation to execute an image processing program prepared in advance. The image processing program may be distributed via a network such as the Internet. Further, the image processing program may be recorded on a computer-readable non-transitory recording medium such as a hard disk, a flexible disk (FD), Compact Disk Read-Only Memory (CD-ROM), a Magneto-Optical (MO) disk, a Digital Versatile Disk (DVD), or the like, so as to be executed as being read from the recording medium by a computer.

According to at least one aspect of the embodiments described above, the operator is able to easily perform the measuring processes by using the ultrasound image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising processing circuitry configured:
to generate an ultrasound image on a basis of a result of an ultrasound scan performed on a region including a part of a subject;
to obtain a schematic image schematically indicating the part of the subject; and
to cause a display to display the schematic image and either the ultrasound image or an image based on the ultrasound image, in such a manner that an orientation of the subject included in either the ultrasound image or the image based on the ultrasound image and an orientation of the subject indicated in the schematic image are close to each other, on a basis of an analysis result from an analysis performed on either the ultrasound image or the image based on the ultrasound image.

2. The ultrasound diagnosis apparatus according to claim 1, wherein the processing circuitry causes the display to display the schematic image and either the ultrasound image or the image based on the ultrasound image, in such a manner that the orientation of the subject included in either the ultrasound image or the image based on the ultrasound image is same as the orientation of the subject indicated in the schematic image.

3. The ultrasound diagnosis apparatus according to claim 1, wherein
on the basis of the analysis result, the processing circuitry performs at least one selected from between a rotating process and an inverting process on the schematic image, and
the processing circuitry causes the display to display a schematic image resulting from the process and either the ultrasound image or the image based on ultrasound image.

4. The ultrasound diagnosis apparatus according to claim 3, wherein
every time an ultrasound image is generated, the processing circuitry performs at least one selected from between the rotating process and the inverting process on the schematic image, on the basis of the analysis result, and
every time at least one of the processes is performed, the processing circuitry causes the display to display the schematic image resulting from the process and either the ultrasound image or the image based on the ultrasound image.

5. The ultrasound diagnosis apparatus according to claim 1, wherein
on the basis of the analysis result, the processing circuitry performs at least one selected from between a rotating process and an inverting process on either the ultrasound image or the image based on the ultrasound image, and
the processing circuitry causes the display to display an image resulting from the process and the schematic image.

6. The ultrasound diagnosis apparatus according claim 5, wherein
every time an ultrasound image is generated, the processing circuitry performs at least one selected from between the rotating process and the inverting process on either the ultrasound image or the image based on the ultrasound image, on the basis of the analysis result, and
every time at least one of the processes is performed, the processing circuitry causes the display to display an image resulting from the process and the schematic image.

7. The ultrasound diagnosis apparatus according to claim 1, wherein the subject is a fetus.

8. The ultrasound diagnosis apparatus according to claim 7, wherein the schematic image is an image including information about a measuring method implemented on a part of the fetus.

9. The ultrasound diagnosis apparatus according to claim 1, wherein
the region is a three-dimensional region,
the ultrasound image is a three-dimensional image, and
the image based on the ultrasound image is a tomographic image generated from the three-dimensional image.

10. The ultrasound diagnosis apparatus according to claim 1, wherein
the region is a two-dimensional region, and
the ultrasound image is a tomographic image.

11. The ultrasound diagnosis apparatus according to claim 3, wherein
the processing circuitry performs the analysis on either the ultrasound image or the image based on the ultrasound image, and
on the basis of the analysis result from the analysis, the processing circuitry performs at least one selected from between the rotating process and the inverting process on the schematic image.

12. The ultrasound diagnosis apparatus according to claim 11, wherein
the processing circuitry analyzes an orientation of a structure included in the part of the subject from either the ultrasound image or the image based on the ultrasound image, and
the processing circuitry rotates the schematic image on a basis of the orientation of the structure.

13. The ultrasound diagnosis apparatus according to claim 11, wherein
the processing circuitry analyzes an orientation of a bone included in the part of the subject from either the ultrasound image or the image based on the ultrasound image, and
the processing circuitry rotates the schematic image on a basis of the orientation of the bone.

14. The ultrasound diagnosis apparatus according to claim 11, wherein
from either the ultrasound image or the image based on the ultrasound image, the processing circuitry analyzes a positional relationship between an image region indicating the part of the subject and a bone image region indicating a bone included in the part of the subject, and
the processing circuitry inverts the schematic image on the basis of the positional relationship.

15. The ultrasound diagnosis apparatus according to claim 14, wherein the processing circuitry analyzes a positional relationship between a center of gravity of the part of the subject indicated in the image region and a center of gravity of the bone indicated in the bone image region.

16. The ultrasound diagnosis apparatus according to claim 11, wherein
upon detecting, from the ultrasound image, a bone image region indicating a bone included in the part of the subject, the processing circuitry calculates a reliability of the detected bone image region, and
when the reliability is higher than a threshold value, the processing circuitry performs at least one selected from between the rotating process and the inverting process on the schematic image, on the basis of the analysis result from the analysis.

17. The ultrasound diagnosis apparatus according to claim 1, wherein the part of the subject is either an upper arm or a thigh.

18. The ultrasound diagnosis apparatus according to claim 1, wherein the schematic image is represented by vector data.

19. An image processing method comprising:
generating an ultrasound image on a basis of a result of an ultrasound scan performed on a region including a part of a subject;
obtaining a schematic image schematically indicating the part of the subject; and
causing a display to display the schematic image and either the ultrasound image or an image based on the ultrasound image, in such a manner that an orientation of the subject included in either the ultrasound image or the image based on the ultrasound image and an orientation of the subject indicated in the schematic image are close to each other, on a basis of an analysis result from an analysis performed on either the ultrasound image or the image based on the ultrasound image.
